(19) United States
(12) Patent Application Publication
(10) Pub. No.: US 2007/0275996 A1
(43) Pub. Date: Nov. 29, 2007

(54) Use of Statins For The Treatment Of Metabolic Syndrome

(76) Inventor: Michele Bortolini, Basel (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/582,739

(22) PCT Filed: Dec. 15, 2004
(86) PCT No.: PCT/EP04/14304

§ 371 Date: May 10, 2007

Related U.S. Application Data
(60) Provisional application No. 60/529,831, filed on Dec. 16, 2003.

(51) Int. Cl. A61K 31/404

(52) U.S. Cl. 514/311; 514/419

(57) ABSTRACT

The present invention relates to the use of a pharmaceutical composition consisting of a statin (especially fluvastatin or pitavastatin) or a pharmaceutically acceptable salt thereof and a pharmaceutically acceptable carrier for the preparation of a medicament for the prevention or treatment of metabolic syndrome (or syndrome X).

Use of Organic Compounds

The present invention relates to the use of a pharmaceutical composition consisting of a statin (especially fluvastatin or pitavastatin) or a pharmaceutically acceptable salt thereof and a pharmaceutically acceptable carrier for the preparation of a medicament for the prevention or treatment of metabolic syndrome (or syndrome X).

HMG-CoA reductase inhibitors, (also called β-hydroxy-βmethylglutaryl-co-enzyme-A reductase inhibitors and also called statins) are understood to be those active agents which may be preferably used to lower the lipid levels including cholesterol in blood and can be used e.g. for the prevention or treatment of hyperlipidemia and artheriosclerosis.

The class of HMG-Co-A reductase inhibitors comprises compounds having differing structural features.

Preferred are compounds which are selected from the group consisting of atorvastatin, cerivastatin, fluvastatin, lovastatin, pitavastatin (formerly itavastatin), pravastatin, rosuvastatin, and simvastatin, or, in each case, a pharmaceutically acceptable salt thereof.

Especially preferred HMG-Co-A reductase inhibitors are those agents which have been marketed. Most preferred are atorvastatin, fluvastatin, pitavastatin, rosuvastatin or simvastatin or a pharmaceutically acceptable salt thereof, in the first line fluvastatin or pitavastatin or a pharmaceutically acceptable salt thereof.

Only salts that are pharmaceutically acceptable and non-toxic are used therapeutically and those salts are therefore preferred.

Especially preferred are sodium salts of fluvastatin and calcium salts of pitavastatin.

The structure of the active agents identified hereinbefore or hereinafter by generic or trade names may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active agent and, based on these references, likewise enabled to manufacture and test the pharmaceutical indications and properties in standard test models, both in vitro and in vivo.

Metabolic Syndrome

Metabolic syndrome (also called syndrome X) is a complex syndrome which can be associated with several of following criteria such as resistance to insulin-stimulated glucose uptake, glucose intolerance, hyperinsulinemia, increase LDL-cholesterol, increased VLDL triglycerides, decreased HDL cholesterol, increased plasminogen activator inhibitor-1 (PAI-1) levels and hypertension. Other metabolic abnormalities that have been considered as part of the syndrome include abnormal weight or weight distribution, inflammation, microalbuminuria, hyperuricemia, and abnormalities of fibrinolysis and of coagulation.

Glucose intolerance is characterized by a pathological state in which the fasting plasma glucose level is less than 140 mg per deciliter and the 30-, 60-, or 90-minute plasma glucose concentration following a glucose tolerance test exceeds 200 mg per deciliter.

Hyperinsulinemia is a condition in which the level of insulin in the blood is higher than normal. hyperinsulinemia is caused by overproduction of insulin by the body and related to insulin resistance.

very low density lipoprotein ( VLDL) are large lipoproteins rich in triglycerides which circulate through the blood giving up their triglycerides to fat and muscle tissue until the VLDL remnants are modified and converted into LDL.

High density lipoprotein (HDL) are lipoproteins that transport cholesterol in the blood; composed of a high proportion of protein and relatively little cholesterol; high levels are thought to be associated with decreased risk of coronary heart disease and atherosclerosis.

Inflammation is characterized by a response of redness, swelling, pain, and a feeling of heat in certain areas Inflammation is meant to protect tissues affected by injury or disease. There may be loss of function where there is inflammation.

Microalbuminuria is characterized by urinary albumin excretion rate of greater than 20 mcg/min but less than 200 mcg/min on two of three urine samples collected over a six month period. This is approximately 30-300 mg/24 hrs.

Hyperuricemia is characterized by a buildup of uric acid (a byproduct of metabolism) in the blood.

Fibrinolysis is defined as a normal ongoing process that dissolves fibrin and results in the removal of small blood clots.

Hypertriglyceridemia is defined by elevated triglyceride concentration in the blood. Hyperlipidemia is charcterized by the presence of excess lipids in the blood.

Central obesity is characterized by the deposition of obesity around the trunk sparing the limbs.

Body Mass Index (BMI) is a relationship between weight and height that is associated with body fat and health risk.

People with the metabolic syndrome are at increased risk for cardiovascular disease and for increased mortality from both cardiovascular disease and all causes . Studies also have found that clustering of risk factors proposed to be part of the metabolic syndrome may increase the risk for coronary heart disease In addition, components of the metabolic syndrome are risk factors for diabetes .

Among various definition of Metabolic syndrome that are known three of them are of particular relevance:

Metabolic syndrome is characterized by three or more of the following criteria:

1. Abdominal obesity: waist circumference >102 cm in men and >88 cm in women

2. Hypertriglyceridemia: ≧150 mg/dl (1.695 mmol/l)

3. Low HDL cholesterol: <40 mg/dl (1.036 mmol/l) in men and <50 mg/dl (1.295 mmol/l) in women

4. High blood pressure: ≧130/85 mmHg

5. High fasting glucose: ≧110 mg/dl (≧6.1 mmol/l).

Metabolic syndrome can also be characterized by three or more of the following criteria: triglycerides >150 mg/dl, systolic blood pressure (BP) ≧130 mm Hg or diastolic BP ≧85 mm Hg or on antihypertensive treatment, high-density lipoprotein cholesterol <40 mg/dl, fasting blood sugar (FBS) >110 mg/dl, and a BMI >28.8 k/m2.

Metabolic syndrome ca also be characterized by diabetes, impaired glucose tolerance, impaired fasting glucose, or insulin resistance plus two or more of the following abnormalities:

1. High blood pressure: ≧160/90 mmHg

2. Hyperlipidemia: triglyceride concentration ≧150 mg/dl (1.695 mmol/l) and/or HDL cholesterol <35 mg/dl (0.9 mmol/l) in men and <39 mg/dl (1.0 mmol/l) in women

3. Central obesity: waist-to-hip ratio of >0.90 in men or >0.85 in women and/or body mass index (BMI) >30 kg/m2

4. Microalbuminuria: urinary albumin excretion rate ≧90 μg/min or an albumin-to-creatinine ratio ≧90 mg/g.

It is the object of this invention to provide pharmaceutical compositions for the prevention delay of progression or treatment of metabolic syndrome, which composition comprises a statin (especially fluvastatin or pitavastatin) or a pharmaceutically acceptable salt thereof.

The term “Prevention” means prophylactic administration of the combination to healthy patients to prevent the outbreak of the conditions mentioned herein. Moreover, the term “prevention” means prophylactic administration of such combination to patients being in a pre-stage of the conditions, to be treated.

The term “delay of progression” used herein means administration of the combination, such as a combined preparation or pharmaceutical composition, to patients being in a pre-stage of the condition to be treated in which patients a pre-form of the corresponding condition is diagnosed.

By the term “treatment” is understood the management and care of a patient for the purpose of combating the disease, condition, or disorder.

It is another object of this invention to provide pharmaceutical compositions according to the invention for the prevention, delay of progression or treatment of metabolic syndrome, which composition comprises a HMG CoA reductase inhibitor or a pharmaceutically acceptable salt thereof, selected from the group consisting of atorvastatin, cerivastatin, fluvastatin, lovastatin, pitavastatin, pravastatin, rosuvastatin, and simvastatin and a pharmaceutically acceptable carrier..

In an another embodiment this invention provides pharmaceutical compositions according to the invention for the prevention, delay of progression or treatment of metabolic syndrome wherein the metabolic syndrome is associated with resistance to insulin-mediated glucose uptake, glucose intolerance, hyperinsulemia, increased LDL-cholesterol, increased VLDL and triglycerides, decreased HDL-cholesterol, increased plasminogen activator inhibitor-1 (PAI-1) levels and hypertension.

In an another embodiment this invention provides pharmaceutical compositions according to the invention for the prevention, delay of progression or treatment of metabolic syndrome wherein the metabolic syndrome is characterized by three or more of the following criteria:

1. Abdominal obesity: waist circumference >102 cm in men and >88 cm in women

2. Hypertriglyceridemia: ≧150 mg/dl (1.695 mmol/l)

3. Low HDL cholesterol: <40 mg/dl (1.036 mmol/l) in men and <50 mg/dl (1.295 mmol/l) in women

4. High blood pressure: ≧130/85 mmHg

5. High fasting glucose: ≧110 mg/dl (≧6.1 mmol/l)

In an another embodiment this invention provides pharmaceutical compositions according to the invention for the prevention, delay of progression or treatment of metabolic syndrome wherein the metabolic syndrome is characterized by diabetes, impaired glucose tolerance, impaired fasting glucose, or insulin resistance plus two or more of the following abnormalities:

1. High blood pressure: ≧160/90 mmHg

2. Hyperlipidemia: triglyceride concentration ≧150 mg/dl (1.695 mmol/l) and/or HDL cholesterol <35 mg/dl (0.9 mmol/l) in men and <39 mg/dl (1.0 mmol/l) in women

3. Central obesity: waist-to-hip ratio of >0.90 in men or >0.85 in women and/or BMI >30 kg/m2

4. Microalbuminuria: urinary albumin excretion rate ≧20 μg/min or an albumin-to-creatinine ratio ≧90 mg/g.

In an another embodiment this invention provides a pharmaceutical compositions according to the invention for the prevention, delay of progression or treatment of metabolic syndrome wherein the metabolic syndrome is characterized by by three or more of the following: triglycerides >150 mg/dl, systolic blood pressure (BP) ≧130 mm Hg or diastolic BP ≧85 mm Hg or on antihypertensive treatment, high-density lipoprotein cholesterol <40 mg/dl, fasting blood sugar (FBS) >110 mg/dl, and a BMI >28.8 k/m2.

The invention also relates to the use of a pharmaceutical composition according to the invention for the preparation of a medicament for the prevention, delay of progression or treatment of metabolic syndrome.

The invention also relates to the use of a pharmaceutical composition according to the invention for the preparation of a medicament for the prevention, delay of progression or treatment of metabolic syndrome wherein the metabolic syndrome is associated with resistance to insulin-mediated glucose uptake, glucose intolerance, hyperinsulemia, increased LDL-cholesterol, increased VLDL and triglycerides, decreased HDL-cholesterol, increased plasminogen activator inhibitor-1 (PAI-1) levels and hypertension.

The invention also relates to the use of a pharmaceutical composition according to the invention for the preparation of a medicament for the prevention, delay of progression or treatment of metabolic syndrome wherein the metabolic syndrome is characterized by three or more of the following criteria:

1. Abdominal obesity: waist circumference >102 cm in men and >88 cm in women

2. Hypertriglyceridemia: ≧150 mg/dl (1.695 mmol/l)

3. Low HDL cholesterol: <40 mg/dl (1.036 mmol/l) in men and <50 mg/dl (1.295 mmol/l) in women

4. High blood pressure: ≧130/85 mmHg

5. High fasting glucose: ≧110 mg/dl (≧6.1 mmol/l).

The invention also relates to the use of a pharmaceutical composition according to the invention for the preparation of a medicament for the prevention, delay of progression or treatment of metabolic syndrome wherein the metabolic syndrome is characterized by diabetes, impaired glucose tolerance, impaired fasting glucose, or insulin resistance plus two or more of the following abnormalities:

1. High blood pressure: ≧160/90 mmHg

2. Hyperlipidemia: triglyceride concentration ≧150 mg/dl (1.695 mmol/l) and/or HDL cholesterol <35 mg/dl (0.9 mmol/l) in men and <39 mg/dl (1.0 mmol/l) in women

3. Central obesity: waist-to-hip ratio of >0.90 in men or >0.85 in women and/or BMI >30 kg/m2

4. Microalbuminuria: urinary albumin excretion rate ≧90 μg/min or an albumin-to-creatinine ratio ≧90 mg/g.

The invention also relates to the use of a pharmaceutical composition according to the invention for the preparation of a medicament for the prevention, delay of progression or treatment of metabolic syndrome wherein the metabolic syndrome is characterized by three or more of the following: triglycerides >150 mg/dl, systolic blood pressure (BP) ≧130 mm Hg or diastolic BP ≧85 mm Hg or on antihypertensive treatment, high-density lipoprotein cholesterol <40 mg/dl, fasting blood sugar (FBS) >110 mg/dl, and a BODY MASS INDEX (BMI) >28.8 k/m2.

The invention also relates to a method for the prevention, delay of progression or treatment of metabolic syndrome, which method comprises administering to a mammal in need thereof a therapeutically effective amount of a pharmaceutical composition according to the invention.

The invention also relates to a method for the prevention, delay of progression or treatment of metabolic syndrome wherein the metabolic syndrome is associated with resistance to insulin-mediated glucose uptake, glucose intolerance, hyperinsulemia, increased LDL-cholesterol, increased VLDL and triglycerides, decreased HDL-cholesterol, increased plasminogen activator inhibitor-1 (PAI-1) levels and hypertension.

The invention also relates to a method for the prevention, delay of progression or treatment of metabolic syndrome wherein the metabolic syndrome is characterized by three or more of the following criteria:

1. Abdominal obesity: waist circumference >102 cm in men and >88 cm in women

2. Hypertriglyceridemia: ≧150 mg/dl (1.695 mmol/l)

3. Low HDL cholesterol: <40 mg/dl (1.036 mmol/l) in men and <50 mg/dl (1.295 mmol/l) in women

4. High blood pressure: ≧130/85 mmHg

5. High fasting glucose: ≧110 mg/dl (≧6.1 mmol/l) .

The invention also relates to a method for the prevention, delay of progression or treatment of metabolic syndrome wherein the metabolic syndrome is characterized by diabetes, impaired glucose tolerance, impaired fasting glucose, or insulin resistance plus two or more of the following abnormalities:

1. High blood pressure: ≧160/90 mmHg

2. Hyperlipidemia: triglyceride concentration ≧150 mg/dl (1.695 mmol/1) and/or HDL cholesterol <35 mg/dl (0.9 mmol/l) in men and <39 mg/dl (1.0 mmol/l) in women

3. Central obesity: waist-to-hip ratio of >0.90 in men or >0.85 in women and/or body mass index (BMI) >30 kg/m2

4. Microalbuminuria: urinary albumin excretion rate ≧20 μg/min or an albumin-to-creatinine ratio ≧20 mg/g.

The invention also relates to a method for the prevention, delay of progression or treatment of metabolic syndrome wherein the metabolic syndrome is characterized by three or more of the following: triglycerides >150 mg/dl, systolic blood pressure (BP) ≧130 mm Hg or diastolic BP≧85 mm Hg or on antihypertensive treatment, high-density lipoprotein cholesterol <40 mg/dl, fasting blood sugar (FBS) >110 mg/dl, and a BMI >28.8 k/m2.

The present invention relates to a package comprising an HMG CoA reductase inhibitor or a pharmaceutically acceptable salt thereof together with instructions for use for the prevention, delay of progression or treatment of metabolic syndrome.

The present invention relates to a package comprising an HMG CoA reductase inhibitor or a pharmaceutically acceptable salt thereof, especially fluvastatin or pitavastatin together with instructions for use for the prevention, delay of progression or treatment of metabolic syndrome.

Said pharmaceutical compositions are those for enteral, such as oral, and also rectal or parenteral administration to mammals (warm-blooded animals), including man, the pharmacological active ingredient being present on its own or together with the usual pharmaceutical excipients. The pharmaceutical compositions contain, for example, from about 0.1% to 100%, preferably from about 1% to about 80%, of the active ingredient. Pharmaceutical compositions for enteral or parenteral and also for ocular administration are typically those in unit dose forms, such as dragées, tablets, capsules or suppositories and also ampoules. These are prepared in a manner known per se,-for example by means of conventional mixing, granulating, sugar-coating, dissolving or lyophilising methods. Accordingly, pharmaceutical compositions for oral use can be obtained by combining the active ingredient with solid carriers, if desired granulating a mixture obtained, and processing the mixture or granules, if desired or necessary after the addition of suitable excipients, to give tablets or dragée cores.

Suitable carriers are preferably fillers, typically sugars, such as lactose, saccharose, mannitol or sorbitol, cellulose compositions and/or calcium phosphates, e.g. tricalcium phosphate or calciumhydrogen phosphate, furthermore binders, such as starch paste, typically using e.g. corn starch, wheat starch, rice starch or potato starch, gelatin, tragacanth gum, methylcellulose and/or polyvinylpyrrolidone and, if desired, disintegrants, such as the above-mentioned starches, furthermore carboxymethyl starch, crosslinked polyvinylpyrrolidone, agar, alginic acid or a salt thereof, typically sodium alginate. Excipients are primarily flow regulators and lubricants, typically silica gel, talcum, stearic acid or salts thereof, typically magnesium stearate or calcium stearate, and/or polyethylene glycol. Dragée cores are provided with suitable coatings which, if desired, are resistant to gastric juice, using, inter alia, concentrated sugar solutions which optionally contain gum arabic, talcum, polyvinylpyrrolidone, polyethylene glycol and/or titanium dioxide, coating solutions in suitable organic solvents or solvent mixtures or, for the preparation of gastric juice-resistant coatings, solutions of suitable cellulose compositions, typically acetylcellulose phthalate or hydroxypropylmethylcellulose phthalate. Colorants or pigments may be added to the tablets or dragée coatings, for example to identify or indicate different doses of active ingredient.

Other pharmaceutical compositions for oral administration are dry-filled gelatin capsules as well as soft closed capsules made of gelatin and a plasticizer, such as glycerol or sorbitol. The dry-filled capsules may contain the active ingredient in the form of granules, typically in admixture with fillers, such as lactose, binders, such as starches, and/or lubricants, such as talcum or magnesium stearate. In soft capsules, the active ingredient is preferably dissolved or suspended in suitable liquids, such as fatty oils, paraffin oil or liquid polyethylene glycols, and stabilisers can also be added.

Suitable pharmaceutical compositions for rectal administration are typically suppositories consisting of a combination of the active ingredient with a suppository base. Suitable suppository bases are, for example, natural or synthetic triglycerides, paraffin hydrocarbons and higher alkanols. Furthermore, gelatin rectal capsules containing a combination of the active ingredient with a base substance may also be used. Suitable base substances are, for example, liquid triglycerides, polyethylene glycols or paraffin hydrocarbons.

Suitable compositions for parenteral administration are primarily aqueous solutions of an active ingredient in water-soluble form, typically a water-soluble salt, and also suspensions of the active ingredient, such as appropriate oily injection suspensions, using suitable lipophilic solvents or vehicles, typically fatty oils, e.g. sesame oil, or synthetic fatty acid esters, typically ethyl oleate or triglycerides, or aqueous injection suspensions containing viscosity-increasing substances, e.g. sodium carboxymethylcellulose, sorbitol and/or dextran and, optionally, also stabilisers.

For preventive treatments, unit dosage forms for oral administration are preferred, typically tablets or capsules and, in acute treatments, i.v. application forms. The dosage of the active compound can depend on a variety of factors, such as mode of administration, homeothermic species, age and/or individual condition.

Preferred dosages for the active ingredients of the pharmaceutical combination according to the present invention are therapeutically effective dosages, especially those which are commercially available.

Normally, in the case of oral administration, an approximate daily dose of from about 1 mg to about 360 mg is to be estimated e.g. for a patient of approximately 75 kg in weight.

The dosage of the active compound can depend on a variety of factors, such as mode of administration, homeothermic species, age and/or individual condition.

In case of HMG-CoA reductase inhibitors, preferred dosage unit forms of HMG-CoA reductase inhibitors are, for example, tablets or capsules comprising e.g. from about 5 mg to about 120 mg, preferably, when using fluvastatin, for example, 20 mg, 40 mg or 80 mg (equivalent to the free acid) of fluvastatin, for example, administered once a day. When using pitavastatin or pharmaceutically acceptable salt preferred dosage unit forms are, for example, tablets or capsules comprising e.g. from about 1-32 mg of pitavastatin or pharmaceutically acceptable salt.

The following Example illustrates the above invention without, however, limiting it in its scope in any way.

### EXAMPLE OF FLUVASTATIN FORMULATION

### Example 1

Hard Gelatin Capsule:

| Component | Amount per unit [mg] |
|---|---|
| Capsule | |
| Fluvastatin Sodium¹⁾ | 21.481²⁾ |
| Calcium Carbonate | 62.840 |
| Sodium Bicarbonate | 2.000 |
| Microcrystalline Cellulose | 57.220 |
| Pregelatinized Starch | 41.900 |
| Purified Water³⁾ | Q.S. |
| Magnesium Stearate | 1.050 |
| Talc | 9.430 |
| Target Capsule Fill Weight | 195.92 |
| Capsule Shell | |
| Hard gelatin Capsule Shell | 48.500 |
| Branding Ink (pre-printed) | |
| White Ink | Trace |
| Red Ink | Trace |
| Target Capsule Weight | 244.42 |

¹⁾includes a 2% overage for moisture
²⁾20 mg of free acid is equivalent to 21.06 mg Na salt
³⁾partially removed during processing

### Example 2

Hard Gelatin Capsule

| Component | Amount per unit [mg] |
|---|---|
| Fluvastatin Sodium | 42.962¹⁾²⁾ |
| Calcium Carbonate | 125.680 |
| Sodium Bicarbonate | 4.000 |
| Microcrystalline Cellulose | 114.440 |
| Pregelatinized Starch | 83.800 |
| Purified Water³⁾ | Q.S. |
| Magnesium Stearate | 2.100 |
| Talc | 18.860 |
| Target Capsule Fill Weight | 391.840 |
| Capsule Shell | |
| Hard gelatin Capsule Shell | 76.500 |
| Branding Ink (pre-printed) | |
| White Ink | Trace |
| Red Ink | Trace |
| Target Capsule Weight | 468.34 |

¹⁾includes a 2% overage for moisture
²⁾20 mg of free acid equivalent to 21.06 mg Na salt
³⁾partially removed during processing

### Example 3

Round, Slightly Bi-convex, Film-Coated Tablets with Beleved Edges:

| Component | Amount per unit [mg] |
|---|---|
| Table Core | |
| Fluvastatin Sodium¹⁾ | 84.24²⁾ |
| Cellulose Microcrystalline/Micro- | 111.27 |
| crystalline cellulose fine powder | |
| Hypromellose/Hydroxypropyl | 97.50 |
| methyl cellulose (Methocel | |
| K100LVP CR; HPMC100 cps) | |
| Hydroxypropyl cellulose (Klucel | 16.25 |
| HXF) | |
| Potassium hydrogen carbonate/ | 8.42 |
| Potassium bicarbonate | |
| Povidone | 4.88 |
| Magnesium stearate | 2.44 |
| Core Tablet Weight | 325.00 |
| Coating | |
| Coating premix - Opadry Yellow | 9.75 |
| (00F22737) | 0 |
| Total Weight | 334.75 |
| Water, purified³⁾ | Q.S. |

¹⁾84.24 mg of the sodium salt of fluvastatin is equivalent to 80 mg of fluvastatin free acid
²⁾to be adjusted for moisture (LOD)
³⁾removed during processing

### Example 4

160mg Enteric-Coated Tablet

| Component | Amount |
|---|---|
| Fluvastatin | 168.48 |
| KHCO₃ | 8.42 |
| Mic. Cryst. Cell. fine powder NF | 65.00 |
| Polyvinylpyrrolidone K30 PH USP | 20.50 |
| Hydroxypropyl-cellulose HXF, NF | 20.50 |
| HPMC K100 LVCR, USP | 110.70 |
| HPMC K4MP CR | 12.30 |
| Mg Stearate, NF | 4.10 |
| | 410.00 |
| Opadry Clear YS-1-19012 | 10.00 |
| Eudragit L-30D-55 | 18.70 |
| Talc | 4.50 |
| PEG 4000, EP | 1.80 |
| Simethicone Emulsion USP | q.s |
| | 445.00 |

### EXAMPLES OF PITAVASTATIN FORMULATIONS

### Example 5 (4MG)

Core (percentage related to core weight): 4.18 mg (5.225% wt) of drug substance, for example pitavastatin Ca-salts, 42.82 mg (53.525% wt) of microcrystalline cellulose, 4 mg (5% wt) of HPMC (3 cps), 25 mg (31.25% wt) of HPMC (100 cps), 3.2 mg (4% wt) of Neusilin, the external phase comprising 0.4 mg (0.5% wt) of silicium dioxide colloidal and 0.4 mg (0.5% wt) of magnesium stearate. HPMC subcoat (non functional coat) (percentage related to subcoat weight): 2.856 mg (71.4% wt) of Hydroxypropylmethylcellulose 3cps, 0.286 mg (7.15% wt) of polyethyleneglycol, 0.286 mg (7.15% wt) of talc and 0.572 mg (14.3% wt) of titanium dioxide.

Enteric coat (percentage related to enteric coat weight): 5 mg (83.34% wt) of Eudragit L30D, 0.5 mg (8.33% wt) of talc and 0.5 mg (8.33% wt) of polyethyleneglycol.

### Example 6 (8MG)

Core (percentage related to core weight): 8.36 mg (10.45% wt) of drug substance, for example pitavastatin Ca-salts, 38.64 mg (48.3% wt) of microcrystalline cellulose, 4 mg (5% wt) of HPMC (3 cps), 25 mg (31.25% wt) of HPMC (100 cps), 3.2 mg (4% wt) of Neusilin, the external phase comprising 0.4 mg (0.5% wt) of silicium dioxide colloidal and 0.4 mg (0.5% wt) of magnesium stearate. HPMC subcoat (non functional coat) (percentage related to subcoat weight): 2.856 mg (71.4% wt) of Hydroxypropylmethylcellulose 3cps, 0.286 mg (7.15% wt) of polyethyleneglycol, 0.286 mg (7.15% wt) of talc and 0.572 mg (14.3% wt) of titanium dioxide.

Enteric coat (percentage related to enteric coat weight): 5 mg (83.34% wt) of Eudragit L30D, 0.5 mg (8.33% wt) of talc and 0.5 mg (8.33% wt) of polyethyleneglycol.

### Example 7 (16MG)

Core (percentage related to core weight): 16.72 mg (20.9% wt) of drug substance, for example pitavastatin Ca-salts, 30.28 mg (37.85% wt) of microcrystalline cellulose, 4 mg (5% wt) of HPMC (3 cps), 25 mg (31.25% wt) of HPMC (100 cps), 3.2 mg (4% wt) of Neusilin, the external phase comprising 0.4 mg (0.5% wt) of silicium dioxide colloidal and 0.4 mg (0.5% wt) of magnesium stearate.

HPMC subcoat (non functional coat) (percentage related to subcoat weight): 2.856 mg (71.4% wt) of Hydroxypropylmethylcellulose 3cps, 0.286 mg (7.15% wt) of polyethyleneglycol, 0.286 mg (7.15% wt) of talc and 0.572 mg (14.3% wt) of titanium dioxide.

Enteric coat (percentage related to enteric coat weight): 5 mg (83.34% wt) of Eudragit

L30D, 0.5 mg (8.33% wt) of talc and 0.5 mg (8.33% wt) of polyethyleneglycol.

1-6. (canceled)

7. A pharmaceutical composition for the prevention or treatment of metabolic syndrome consisting of fluvastatin or pitavastatin or a pharmaceutically acceptable salt thereof and a pharmaceutically acceptable carrier.

8. Pharmaceutical composition according to claim 7 wherein the metabolic syndrome is associated with resistance to insulin-mediated glucose uptake, glucose intolerance, hyperinsulemia, increased LDL-cholesterol, increased VLDL and triglycerides, decreased HDL-cholesterol, increased plasminogen activator inhibitor-1 (PAI-1) levels and hypertension.

9. Pharmaceutical composition according to claim 7 wherein the metabolic syndrome is characterized by three or more of the following criteria:
1. Abdominal obesity: waist circumference >102 cm in men and >88 cm in women
2. Hypertriglyceridemia: ≧150 mg/dl (1.695 mmol/l)
3. Low HDL cholesterol: <40 mg/dl (1.036 mmol/l) in men and <50 mg/dl (1.295 mmol/l) in women
4. High blood pressure: ≧130/85 mmHg
5. High fasting glucose: ≧110 mg/dl (6.1 mmol/l).

10. Pharmaceutical composition according to claim 7 wherein the metabolic syndrome is characterized by diabetes, impaired glucose tolerance, impaired fasting glucose, or insulin resistance plus two or more of the following abnormalities:
1 High blood pressure: ≧160/90 mmHg
2 Hyperlipidemia: triglyceride concentration ≧150 mg/dl (1.695 mmol/l) and/or HDL cholesterol <35 mg/dl (0.9 mmol/l) in men and <39 mg/dl (1.0 mmol/l) in women
3 Central obesity: waist-to-hip ratio of >0.90 in men or >0.85 in women and/or BMI >30 kg/m2
4 Microalbuminuria: urinary albumin excretion rate ≧20 μg/min or an albumin to creatinine ratio ≧20 mg/g.

11. Pharmaceutical composition according to claim 7 wherein the metabolic syndrome is characterized by three or more of the following criteria:
triglycerides >150 mg/dl, systolic blood pressure (BP) ≧130 mm Hg or diastolic BP ≧85 mm Hg or on antihypertensive treatment, high-density lipoprotein cholesterol <40 mg/dl, fasting blood sugar (FBS) >110 mg/dl, and a BMI >28.8 k/m2.

12. A method for the prevention or treatment of metabolic syndrome comprising in administering a pharmaceutical composition to the patient, wherein the pharmaceutical composition consists of fluvastatin or pitavastatin or a pharmaceutically acceptable salt thereof and a pharmaceutically acceptable carrier.

13. Method according to claim 12 wherein the metabolic syndrome is associated with resistance to insulin-mediated glucose uptake, glucose intolerance, hyperinsulemia, increased LDL-cholesterol, increased VLDL and triglycerides, decreased HDL-cholesterol, increased plasminogen activator inhibitor-1 (PAI-1) levels and hypertension.

14. Method according to claim 12 wherein the metabolic syndrome is characterized by three or more of the following criteria:
1. Abdominal obesity: waist circumference >102 cm in men and >88 cm in women
2. Hypertriglyceridemia: ≧150 mg/dl (1.695 mmol/l)
3. Low HDL cholesterol: <40 mg/dl (1.036 mmol/l) in men and <50 mg/dl (1.295 mmol/l) in women
4. High blood pressure: ≧130/85 mmHg
5. High fasting glucose: ≧110 mg/dl (>6.1 mmol/l).

15. Method according to claim 12 wherein the metabolic syndrome is characterized by diabetes, impaired glucose tolerance, impaired fasting glucose, or insulin resistance plus two or more of the following abnormalities:
1 High blood pressure: ≧160/90 mmHg
2 Hyperlipidemia: triglyceride concentration ≧150 mg/dl (1.695 mmol/l) and/or HDL cholesterol <35 mg/dl (0.9 mmol/l) in men and <39 mg/dl (1.0 mmol/l) in women
3 Central obesity: waist-to-hip ratio of >0.90 in men or >0.85 in women and/or BMI >30 kg/m2
4 Microalbuminuria: urinary albumin excretion rate ≧20 μg/min or an albumin to creatinine ratio ≧20 mg/g.

16. Method according to claim 12 wherein the metabolic syndrome is characterized by three or more of the following criteria:
triglycerides >150 mg/dl, systolic blood pressure (BP) ≧130 mm Hg or diastolic BP 285 mm Hg or on antibypertensive treatment, high-density lipoprotein cholesterol <40 mg/dl, fasting blood sugar (FBS) >110 mg/dl, and a BMI >28.8 k/m2.
